(19) United States
(12) Patent Application Publication
(10) Pub. No.: US 2023/0062767 A1
(43) Pub. Date: Mar. 2, 2023

(54) SYRINGE PLUNGER ADAPTER

(71) Applicant: Sciore Aesthetics, LLC, South Jordan, UT (US)

(72) Inventors: Jennifer Sciore, South Jordan, UT (US); Edmund Sciore, South Jordan, UT (US); Michael Shane Holt, Sandy, UT (US)

(21) Appl. No.: 17/823,381

(22) Filed: Aug. 30, 2022

Related U.S. Application Data
(60) Provisional application No. 63/239,759, filed on Sep. 1, 2021.

(51) Int. Cl. A61M 5/31; A61M 5/315

(52) U.S. Cl. CPC A61M 5/3137; A61M 5/31511; A61M 2005/3139

(57) ABSTRACT

A syringe plunger adapter can facilitate the aspiration of a syringe. The syringe plunger adapter includes a loop portion that is configured to be worn on the thumb and an adapter portion that is configured to be selectively secured to the plunger flange of a syringe's plunger. With the adapter portion secured to the plunger flange, a clinician, whose thumb is inserted into the loop portion, can aspirate the syringe by simply pulling the thumb away from the syringe.

### CROSS-REFERENCE TO RELATED APPLICATIONS

This application claims the benefit of U.S. Provisional Patent Application No. 63/239,759 which was filed on Sep. 1, 2021.

### BACKGROUND

When performing injections using a syringe, it is oftentimes necessary to aspirate the syringe to determine whether the syringe's needle is positioned within the vasculature. In particular, after inserting the needle subcutaneously, the syringe's plunger can be pulled back to determine if the needle aspirates blood. If not, and assuming the needle is not supposed to be in the vasculature, the injection can then be performed.

### BRIEF SUMMARY

The present invention extends to a syringe plunger adapter that facilitates the aspiration of a syringe. The syringe plunger adapter includes a loop portion that is configured to be worn on the thumb and an adapter portion that is configured to be selectively secured to the plunger flange of a syringe's plunger. With the adapter portion secured to the plunger flange, a clinician, whose thumb is inserted into the loop portion, can aspirate the syringe by simply pulling the thumb away from the syringe.

In some embodiments, the present invention is implemented as a syringe plunger adapter that includes a loop portion that is configured to receive a clinician's thumb and an adapter portion comprising a distal wall that is spaced from the loop portion by opposing sidewalls. The distal wall may include a notch that is configured to receive a plunger when the plunger's flange is positioned in a channel between the distal wall and the loop portion.

In some embodiments, the notch may extend partially through the distal wall.

In some embodiments, the notch may include a center portion and a notch channel.

In some embodiments, the notch channel may have a width that is less than a width or diameter of the plunger.

In some embodiments, the channel between the distal wall and the loop may have a height that causes the plunger flange to be squeezed between the distal wall and the loop portion.

In some embodiments, the adapter portion may be configured to flex to enable the plunger flange to pass between the distal wall and the loop portion.

In some embodiments, the distal wall may extend perpendicular to the opposing sidewalls.

In some embodiments, a distance between the loop portion and a center portion of the distal wall may be smaller than a distance between the loop portion and the distal wall at end portions of the distal wall.

In some embodiments, the adapter portion may be formed of a flexible material such that the distal wall is configured to flex away from the loop portion when the plunger's flange is inserted between the distal wall and the loop portion.

In some embodiments, the flexible material may be a plastic.

In some embodiments, the channel may extend fully between the distal wall and the loop portion.

In some embodiments, the loop portion may be continuous.

In some embodiments, the present invention may be implemented as a method for using a syringe. A syringe plunger adapter may be obtained. The syringe adapter may include a loop portion that is configured to receive a clinician's thumb and an adapter portion comprising a distal wall that is spaced from the loop portion by opposing sidewalls. The distal wall may include a notch that is configured to receive a plunger when the plunger's flange is positioned in a channel between the distal wall and the loop portion. The syringe plunger adapter may then be attached to a syringe by inserting a plunger of the syringe into the notch.

In some embodiments, a thumb may be inserted into the loop portion and used to apply a pulling force on the loop portion to thereby aspirate the syringe.

In some embodiments, inserting the plunger of the syringe into the notch may include flexing the distal wall away from the loop portion.

In some embodiments, inserting the plunger of the syringe into the notch may include flexing a notch channel to allow the syringe to pass into a center portion of the notch.

In some embodiments, the present invention may be implemented as a syringe plunger adapter that includes a loop portion that is configured to receive a clinician's thumb and an adapter portion comprising a distal wall that is spaced from the loop portion by opposing sidewalls. The distal wall may include a notch. The distal wall may be configured to flex away from the loop portion to receive a flange of a syringe's plunger.

In some embodiments, the notch may be positioned in a center portion of the distal wall.

In some embodiments, the distal wall may be perpendicular to the opposing sidewalls.

In some embodiments, a distance between the loop portion and a center portion of the distal wall may be smaller than a distance between the loop portion and the distal wall at end portions of the distal wall.

This summary is provided to introduce a selection of concepts in a simplified form that are further described below in the Detailed Description. This Summary is not intended to identify key features or essential features of the claimed subject matter.

### BRIEF DESCRIPTION OF THE DRAWINGS

Understanding that these drawings depict only typical embodiments of the invention and are not therefore to be considered limiting of its scope, the invention will be described and explained with additional specificity and detail through the use of the accompanying drawings in which:

FIGS. 1A-1F provide various views of a syringe loop that is configured in accordance with one or more embodiments of the present invention; and

FIG. 2 provides an example of how a syringe loop may facilitate aspiration of a syringe.

### DETAILED DESCRIPTION

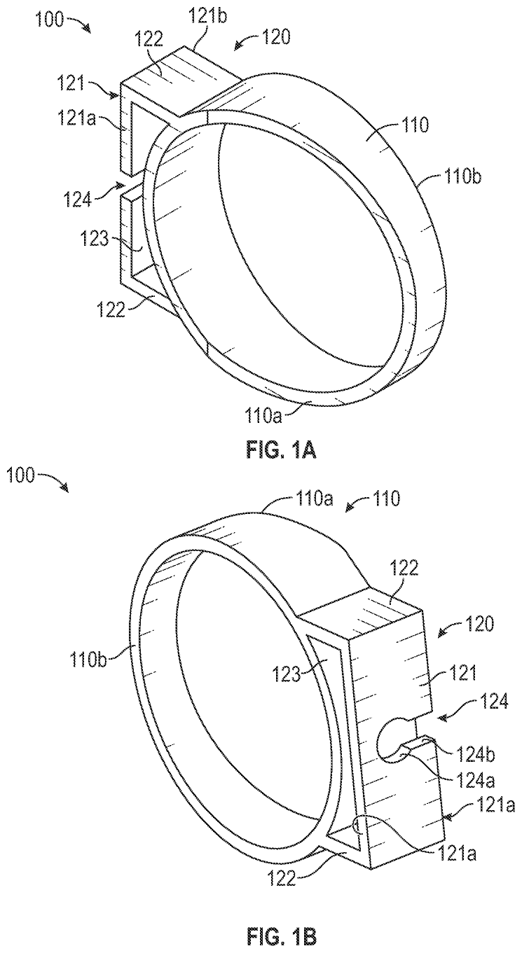

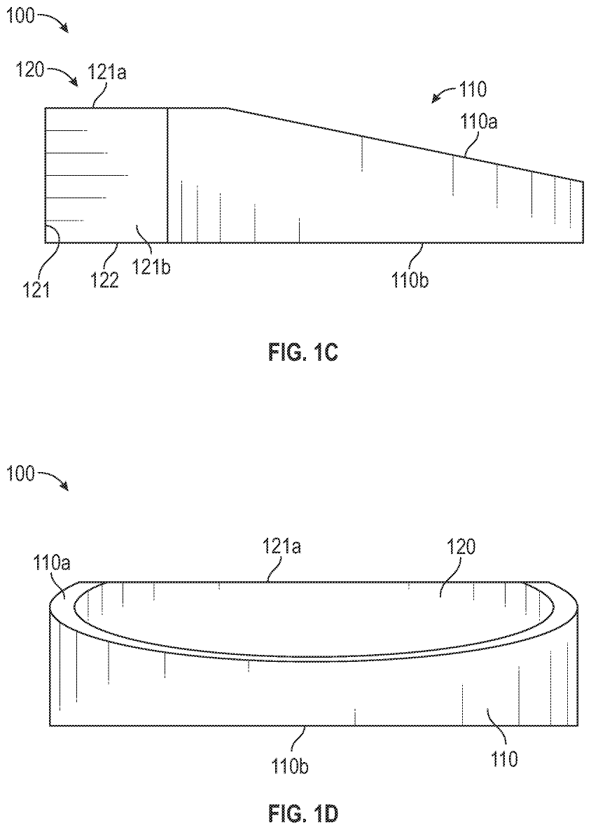

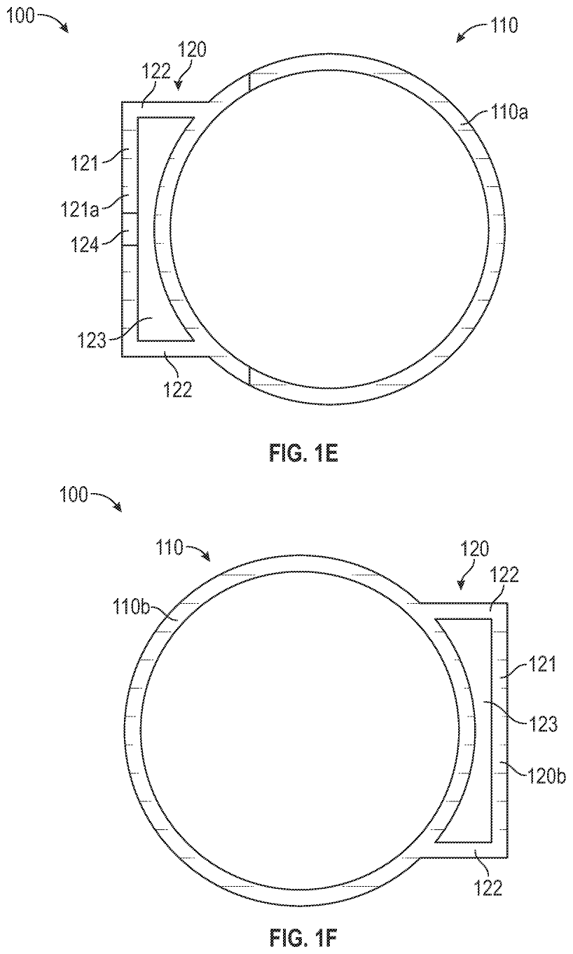

FIGS. 1A-1F provide various views of a syringe plunger adapter 100 that is configured in accordance with one or more embodiments of the present invention. Syringe plunger adapter 100 includes a loop portion 110 and an adapter portion 120 that extends outwardly from loop portion 110. Loop portion 110 may be in the form of a ring and may be sized to fit around the thumb of a clinician. Although loop portion 110 is shown as forming a complete loop or ring, in some embodiments, loop portion 110 may form a partial loop or ring or another structure that ensures that loop portion 110 can remain on a clinician's thumb during use. In some embodiments, a first side 110a of loop portion 110 may be tapered while a second side 110b of loop portion 110 may be flat or planar. As a result, a width of loop portion 110 may be greater towards adapter portion 120. In some embodiments, syringe plunge adapter 100 may be formed of a semi-rigid plastic material.

Adapter portion 120 can include a distal wall 121 that is spaced away from loop portion 110 by opposing sidewalls 122 thereby forming a channel 123 between distal wall 121 and loop portion 110. In some embodiments, distal wall 121 may extend perpendicular to opposing sidewalls 122. In some embodiments, channel 123 may pass fully through adapter portion 120. A notch 124 may be formed in a first side 121a of distal wall 121 and may extend into a center of distal wall 121. Notch 124 may include a center portion 124a and a notch channel 124b.

In some embodiments, notch channel 124b may have a width that is less than a width/diameter of a plunger of a syringe, while center portion 124a may have a width/diameter that substantially matches or is slightly less than the width/diameter of the plunger. Channel 123 may have a height that substantially matches or is slightly less than the thickness of the plunger flange. In such embodiments, when the plunger flange is inserted into channel 123 and the plunger is inserted into notch channel 124b, distal wall 121 and/or sidewalls 122 can flex sufficiently to allow the plunger to be inserted into center portion 124a where it can be held snug. Likewise, the plunger flange can be held snug between distal wall 121 and loop portion 110. Therefore, when adapter portion 120 is coupled to the plunger, there will be minimal relative movement between the plunger and syringe plunger adapter 100.

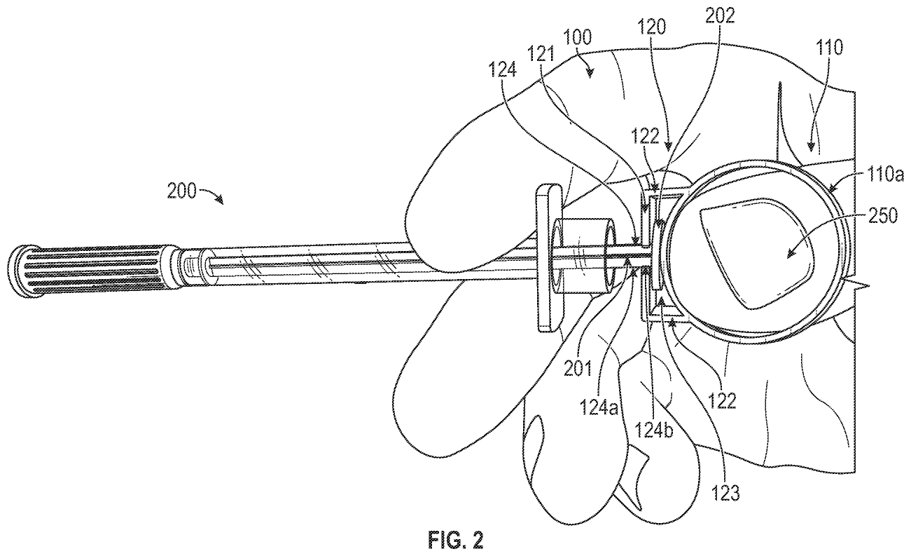

FIG. 2 provides an example of how syringe plunger adapter 100 may be connected to a plunger 201 of a syringe 200. As shown, plunger flange 202 at the proximal end of plunger 201 in positioned within channel 123 and plunger 201 is positioned within center portion 124a. Notch channel 124b is narrower than plunger 201 such that plunger 201 will not escape from notch 124 absent substantial, and likely intentional, force. A distance between loop portion 110 and a center portion of distal wall 121 (e.g., where notch 124 is located) is smaller than a distance between loop portion 110 and distal wall 121 at end portions of distal wall 121 (e.g., where distal wall 121 and sidewalls 122 connect). A clinician may insert his or her thumb 250 to a desired depth within loop portion 110. Then, the clinician may aspirate syringe 200 by simply pulling thumb 250 away from syringe 200.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

What is claimed:

1. A syringe plunger adapter comprising:
a loop portion that is configured to receive a clinician's thumb; and
an adapter portion comprising a distal wall that is spaced from the loop portion by opposing sidewalls, the distal wall including a notch that is configured to receive a plunger when the plunger's flange is positioned in a channel between the distal wall and the loop portion.

2. The syringe plunger adapter of claim 1, wherein the notch extends partially through the distal wall.

3. The syringe plunger adapter of claim 2, wherein the notch includes a center portion and a notch channel.

4. The syringe plunger adapter of claim 3, wherein the notch channel has a width that is less than a width or diameter of the plunger.

5. The syringe plunger adapter of claim 1, wherein the channel between the distal wall and the loop has a height that causes the plunger flange to be squeezed between the distal wall and the loop portion.

6. The syringe plunger adapter of claim 5, wherein the adapter portion is configured to flex to enable the plunger flange to pass between the distal wall and the loop portion.

7. The syringe plunger adapter of claim 1, wherein the distal wall extends perpendicular to the opposing sidewalls.

8. The syringe plunger adapter of claim 1, wherein a distance between the loop portion and a center portion of the distal wall is smaller than a distance between the loop portion and the distal wall at end portions of the distal wall.

9. The syringe plunger adapter of claim 8, wherein the adapter portion is formed of a flexible material such that the distal wall is configured to flex away from the loop portion when the plunger's flange is inserted between the distal wall and the loop portion.

10. The syringe plunger adapter of claim 9, wherein the flexible material is a plastic.

11. The syringe plunger adapter of claim 1, wherein the channel extends fully between the distal wall and the loop portion.

12. The syringe plunger adapter of claim 1, wherein the loop portion is continuous.

13. A method for using a syringe comprising:
obtaining a syringe plunger adapter comprising:
a loop portion that is configured to receive a clinician's thumb; and
an adapter portion comprising a distal wall that is spaced from the loop portion by opposing sidewalls, the distal wall including a notch that is configured to receive a plunger when the plunger's flange is positioned in a channel between the distal wall and the loop portion;
attaching the syringe plunger adapter to a syringe by inserting a plunger of the syringe into the notch.

14. The method of claim 13, further comprising:
inserting a thumb into the loop portion; and
using the thumb to apply a pulling force on the loop portion to thereby aspirate the syringe.

15. The method of claim 13, wherein inserting the plunger of the syringe into the notch comprises flexing the distal wall away from the loop portion.

16. The method of claim 13, wherein inserting the plunger of the syringe into the notch comprises flexing a notch channel to allow the syringe to pass into a center portion of the notch.

17. A syringe plunger adapter comprising:
a loop portion that is configured to receive a clinician's thumb; and
an adapter portion comprising a distal wall that is spaced from the loop portion by opposing sidewalls, the distal wall including a notch, wherein the distal wall is configured to flex away from the loop portion to receive a flange of a syringe's plunger.

18. The syringe plunger adapter of claim 17, wherein the notch is positioned in a center portion of the distal wall.

19. The syringe plunger adapter of claim 18, wherein the distal wall is perpendicular to the opposing sidewalls.

20. The syringe plunger adapter of claim 17, wherein a distance between the loop portion and a center portion of the distal wall is smaller than a distance between the loop portion and the distal wall at end portions of the distal wall.
